SEQUENCE LISTING (26 sequences)

SEQ ID NO: 1 - DNA, 36 bp, Artificial Sequence
  synthetic
  misc_feature (34)..(36): n is a, c, g, or t
acactctttccctacacgacgctgttccgatctnnn

SEQ ID NO: 2 - DNA, 13 bp, Artificial Sequence
  synthetic
agatcggaacagc

SEQ ID NO: 3 - DNA, 35 bp, Artificial Sequence
  synthetic
  misc_feature (33)..(35): n is a, c, g, or t
gtgactggagttcagacgtgtgctcttcacgannn

SEQ ID NO: 4 - DNA, 13 bp, Artificial Sequence
  synthetic
tcgtgaagagcac

SEQ ID NO: 5 - DNA, 34 bp, Artificial Sequence
  synthetic
  misc_feature (32)..(34): n is a, c, g, or t
acactctttccctacacgacgctcttcatcgnnn

SEQ ID NO: 6 - DNA, 13 bp, Artificial Sequence
  synthetic
cgatgaagagcgt

SEQ ID NO: 7 - DNA, 35 bp, Artificial Sequence
  synthetic
  misc_feature (33)..(35): n is a, c, g, or t
gtgactggagttcagacgtgtgctcttcagcannn

SEQ ID NO: 8 - DNA, 13 bp, Artificial Sequence
  synthetic
tgctgaagagcac

SEQ ID NO: 9 - DNA, 34 bp, Artificial Sequence
  synthetic
  misc_feature (32)..(34): n is a, c, g, or t
acactctttccctacacgacgctcttcatgcnnn

SEQ ID NO: 10 - DNA, 13 bp, Artificial Sequence
  synthetic
gcatgaagagcgt

SEQ ID NO: 11 - DNA, 35 bp, Artificial Sequence
  synthetic
  misc_feature (33)..(35): n is a, c, g, or t
gtgactggagttcagacgtgtgctcttcagacnnn

SEQ ID NO: 12 - DNA, 13 bp, Artificial Sequence
  synthetic
tcgtgaagagcac

SEQ ID NO: 13 - DNA, 34 bp, Artificial Sequence
  synthetic
  misc_feature (32)..(34): n is a, c, g, or t
acactctttccctacacgacgctcttcagtcnnn

SEQ ID NO: 14 - DNA, 13 bp, Artificial Sequence
  synthetic
gactgaagagcgt

SEQ ID NO: 15 - DNA, 35 bp, Artificial Sequence
  synthetic
  misc_feature (33)..(35): n is a, c, g, or t
gtgactggagttcagacgtgtgctcttcacagnnn

SEQ ID NO: 16 - DNA, 13 bp, Artificial Sequence
  synthetic
ctgtgaagagcac

SEQ ID NO: 17 - DNA, 34 bp, Artificial Sequence
  synthetic
  misc_feature (32)..(34): n is a, c, g, or t
acactctttccctacacgacgctcttcactgnnn

SEQ ID NO: 18 - DNA, 13 bp, Artificial Sequence
  synthetic
cagtgaagagcgt

SEQ ID NO: 19 - DNA, 37 bp, Artificial Sequence
  synthetic
  misc_feature (35)..(37): n is a, c, g, or t
gtgactggagttcagacgtgtgctgttccgatctnnn

SEQ ID NO: 20 - DNA, 13 bp, Artificial Sequence
  synthetic
agatcggaacagc

SEQ ID NO: 21 - DNA, 23 bp, Artificial Sequence
  synthetic
acactctttccctacacgacgct

SEQ ID NO: 22 - DNA, 24 bp, Artificial Sequence
  synthetic
gtgactggagttcagacgtgtgct

SEQ ID NO: 23 - DNA, 23 bp, Artificial Sequence
  synthetic
acactctttccctacacgacgct

SEQ ID NO: 24 - DNA, 24 bp, Artificial Sequence
  synthetic
gtgactggagttcagacgtgtgct

SEQ ID NO: 25 - DNA, 48 bp, Artificial Sequence
  synthetic
aatgatacggcgaccaccgagatctacactctttccctacacgacgct

SEQ ID NO: 26 - DNA, 64 bp, Artificial Sequence
  synthetic
  misc_feature (25)..(30): n is a, c, g, or t
caagcagaagacggcatacgagatnnnnnngtgactggagttcagacgtgtgctcttccgatct

(19) United States
(12) Patent Application Publication
(10) Pub. No.: US 2019/0194648 A1
(43) Pub. Date: Jun. 27, 2019

(54) CONSTRUCTION METHOD FOR SERIAL SEQUENCING LIBRARIES OF RAD TAGS

(71) Applicant: OCEAN UNIVERSITY OF CHINA, Qingdao (CN)

(72) Inventors: Shi WANG, Qingdao (CN); Zhenmin BAO, Qingdao (CN); Pingping LIU, Qingdao (CN); Jia LV, Qingdao (CN); Lingling ZHANG, Qingdao (CN)

(21) Appl. No.: 15/741,755

(22) PCT Filed: Jul. 12, 2017
(86) PCT No.: PCT/CN2017/092556

§ 371 Date: Jan. 3, 2018

(30) Foreign Application Priority Data
Aug. 2, 2016 (CN) 201610629494.9

(51) Int. Cl. C12N 15/10

(52) U.S. Cl. CPC C12N 15/1065; C40B 50/06; C12N 15/1068

(57) ABSTRACT

The present invention discloses a construction method for serial sequencing libraries of RAD tags, including the following steps: conducting an enzyme digestion reaction with DNA using endonuclease; ligating; enzyme-digested fragments with adaptors that contain restriction enzyme sites of SapI, featured base sequences for serial ligation of RAD tags, and universal sequences for the binding of amplification primers; conducting PCR amplification using a combination of biotin primers and general primers; collecting target PCR product by gel; amplifying again; and equally mixing and purifying; conducting enzyme digestion on the PCR products using the SapI enzyme and heating tags in series; purifying the long serial tags through gels and then conducting PCR. amplification using the barcode primers for constructing the libraries; and library sequencing, The present invention can be applied to the screening and detection of genome-wide genetic markers and epigenetic variations at high throughput and low cost

### TECHNICAL FIELD

The present invention belongs to the technical field of the detection of DNA genetic markers and DNA methylation in molecular biology, and it especially relates to a construction method for serial sequencing libraries of RAD tags.

### BACKGROUND

In recent years, the rapid development of high-throughput sequencing technology greatly promotes the depth and breadth of studies on animal and plant genomics. A Reduced-Representation Sequencing technology was developed for genome-wide genotyping at minimal labor and cost. Because sequences that correspond to enzyme-digested fragments of certain sizes are used as some representatives of whole genome sequences, the technology reduces the genome complexity, realizes low cost and is independent of reference genome information. These advantages make it possible to develop omics analysis on non-model organisms that are relatively lacking of genome information. The technology is widely applied to the construction of genetic maps, quantitative trait locus (QTL) mapping, population genetics analysis, phylogenetic analysis, genome assembly and other studies. At present, a restriction-site-associated DNA sequencing (RAD-seq) technology is a representative technology in the held. However, RAD technology has a complicated library construction process and produces unequal lengths of fragments, which could have a bias during construction and sequencing, and many improvement technologies have emerged as progress over time has demanded. A 2b-RAD technology based on IIB type restriction DNA endonucleases can produce isolength tags (32-36 bp) and has consistent amplification efficiency. The 2b-RAD technology can not only enhance the typing accuracy rate but also have flexible adjustment of the tag density through selective bases; thus, it is applicable to different study directions and needs and has a broader applications prospect. A MethylRAD technology developed later further extends the application of this technology to the field of epigenetics. The technology allows for quantitative measurement of genome-wide DNA methylation using a Mrr-like family of methylation-dependent restriction enzymes, which can generate isolength gigs.

With the technical innovation and rapid development of the next-generation sequencing platform, a long read length platform has a lower sequencing cost and a wider application than a short read technology on the premise of the same data volume. The limitations of an existing 2b-RAD or MethylRAD technology is that because tags generated by library construction are short (−35 bp), the technology can only be suited to single-end 35-50 bp sequencing and cannot benefit from the gradually increased sequencing capacity (especially the reads length) of current NGS platforms (such as PE100-150 bp sequencing).

In addition, a serial analysis of gene expression (SAGE) technology applied in the field of gene expression analysis is to linked representative tags of transcripts to form long serial molecules that can be cloned into plasmid vectors for sequencing analysis. However, the technology cannot effectively adjust the number of serial tags and the sequential ligation of the tags, and it cannot allow for the serial ligation of more than three tags. Moreover, the sequencing libraries cannot simultaneously allow the typing of SNP's and the detection of methylation.

### SUMMARY

To solve the above problem, the present invention proposes a construction method for serial sequencing libraries of RAD tags that is capable of high-throughput sequencing for serial tags, and it allows the 2b-RAD or MethylRAD technology to be applied to a paired-end sequencing platform. This invention provides a high-throughput and cost-effective method for the screening and detection of genome-wide genetic markers and epigenetic variation.

To achieve the above purpose, the present invention adopts the following technical solution.

A construction method for serial sequencing libraries of RAD tags includes the following steps:

1) enzyme digestion: performing an enzyme digestion reaction with genomic DNA from N samples using selected endonucleases to obtain N parts of enzyme-digested fragments, where N is an integer greater than 2;

2) adaptor ligation: ligating N parts of enzyme digested fragments with adaptors, i.e., N pairs of adaptor pairs are designed to obtain N parts of ligated products, and the adaptors contain the restriction enzyme sites of SapI, featured base sequences for the serial ligation of RAD tags, and a universal sequence for the binding of amplification primers. The sequential ligation of N groups of enzyme-digested fragments are determined according to the added adaptors;

3) amplification of ligated products: conducting PCR amplification on the N parts of ligated products obtained in step 2) using a different combination of biotin primers and general primers; collecting PCR products by gel; amplifying 4-8 cycles using the same method to obtain N parts of enriched. PCR products; and equally mixing the N parts of enriched PCR products and purifying;

4) serial ligation of tag libraries; conducting enzyme digestion on the mixed and purified N parts of the PCR products using the SapI enzyme to excise the universal adaptors and primer sequences on both ends of each enzyme-digested fragment, and the featured base sequences form cohesive ends that enable the N parts of the PCR products to ligate in series; and the sequential ligation of N parts of tag libraries is based on the complementary pairing of the featured sequences on the adaptors.

5) enrichment of ligated serial tags: purifying the serial tags through a gel and then conducting PCR amplification using the barcode primers to construct the serial sequencing libraries of RAD tags

6) library sequencing: sequencing the serial sequencing libraries of the RAD tags on an IIlumina sequencing platform.

To generate isolength (33-35 bp) tags with cohesive ends, the endonuclease in step 1) is one or more of IIB type restriction endonucleases and the Mrr-like family of methylation-dependent restriction enzymes.

To realize sequential head-to-tail ligation of the RAD tags and to provide a primer bonding point for the next amplification and gathering of the serial tags, the adaptors in step 2) have design features with the following properties: taking five pairs of adaptors as an example, five pairs of adaptor combinations are Ada1a and Ada1b, Ada2a and Ada2b, Ada3a and Ada3b, Ada4a and Ada4b, and Ada5a and Ada5b; each adaptor consists of two nucleotide fragments; a base mutation is designed on an enzyme digestion site of SapI in a sequence of adaptors Ada1a and Ada5b and cannot be subjected to enzyme digestion; when enzyme digestion is conducted on the PCR products of five mixed tags by using the SapI enzyme, a universal sequence of adaptors and primers on the Ada1b and Ada5a, Ada2a and Ada2b, Ada3a and Ada3b, and Ada4a and Ada4b are excised, and the three-base featured sequences form cohesive ends on both sides of the five tag fragments; sequential head-to-tail ligation of the five tags is performed according to complementary pairing of the featured sequences, i.e., Ada1b end is ligated with Ada2a end, Ada2b end is ligated with Ada3a end, Ada3b end is ligated with Ada4a end and Ada4b end is ligated with Ada5a end, to form serial tags; and the universal sequence of Adaptors Ada1a and Ada5b on the serial tags is still reserved, thereby providing a primer bonding point for the next amplification and a gathering of the serial tags.

Further, in step 2), the two nucleotide fragments that form Ada1a have the sequences of SEQ ID NO: 1 and SEQ ID NO: 2; the two nucleotide fragments that form Ada1b have the sequences of SEQ ID NO: 3 and SEQ ID NO: 4: the two nucleotide fragments that form Ada2a have the sequences of SEQ ID NO: 5 and SEQ ID NO: 6; the two nucleotide fragments that form Ada2b have the sequences of SEQ ID NO: 7 and SEQ ID NO: 8; the two nucleotide fragments that form Ada3a have the sequences of SEQ ID NO: 9 and SEQ ID NO: 10; the two nucleotide fragments that form Ada1b have the sequences of SEQ ID NO: 11 and SEQ ID NO: 12; the two nucleotide fragments that form Ada4a have the sequences of SEQ ID NO: 13 and SEQ ID NO: 14; the two nucleotide fragments that form Ada4b have the sequences of SEQ ID NO: 15 and SEQ ID NO: 16; the two nucleotide fragments that form Ada5a have the sequences of SEQ ID NO: 17 and SEQ ID NO: 18; and the two nucleotide fragments that form Ada5b have the sequences of SEQ ID NO: 19 and SEQ ID NO: 20.

To separate the target tag fragment from the universal primer fragments excised by the SapI enzyme in the subsequent purification process and achieve a higher efficiency of the serial ligation of the tags in step 3), there is selection of a combination of biotin primers and general primers that correspond to the adaptor combinations in step 2); taking five pairs of adaptors as an example, the enzyme-digested fragments ligated with Adaptor 1 are amplified using primers Prim1 and BioPrim1; the enzyme-digested fragments ligated with Adaptors 2, 3 and 4 are amplified using primers BioPrim1 and BioPrim2; and the enzyme-digested fragments ligated with Adaptor 5 are amplified using the primers BioPrim1 and Prim2.

Further, the nucleotide sequence of Prim1 is SEQ ID NO: 21; the, nucleotide sequence of Prim2 is SEQ ID NO: 22; the nucleotide sequence of BioPrim1 is SEQ ID NO: 23; and the nucleotide sequence of BioPrim2 is SEQ ID NO: 24.

To enable the serial tag libraries structure to be compatible with the sequencing platform, the primer Barcode is further used to amplify the serial tags; barcodes are introduced for constructing the sequencing libraries, to have a sequencing primer binding site that is compatible on a next-generation sequencing platform, and the nucleotide sequences of the primers in step 5) are SEQ ID NO: 25 and SEQ ID NO: 26.

Compared with the prior art, the present invention has advantages and positive effects with the following aspects: the present invention establishes a construction method for serial sequencing libraries of RAD tags by redesigning the adaptors based on 2b-RAD and MethylRAD technologies, adjusting the corresponding experimental steps and reaction systems for constructing libraries, adding a one-step enzyme digestion ligation reaction and so on. Isolength RAD tags that are generated by 2b-RAD or MethylRAD can be ligated in series to form long fragments to be suitable for paired-end sequencing (e.g., Illumina PE100-150 bp sequencing), which helps to effectively reduce the library constructing cost and sequencing cost, where the library constructing cost is reduced by 20% and the sequencing cost is reduced to 1/10 of the original cost.

In addition, the configuration of the five concatenated tags is highly flexible, and it can be defined by users to work with a desired combination of samples and/or restriction enzymes to suit specific research purposes. Combinations of multienzyme libraries increase the genomic tag density while reducing the cost. Therefore, the present invention provides an efficient and flexible method for screening and detecting genome-wide genetic variations and epigenetic variation.

### DESCRIPTION OF THE DRAWINGS

FIG. 1 shows the procedure of the Multi-isoRAD method.

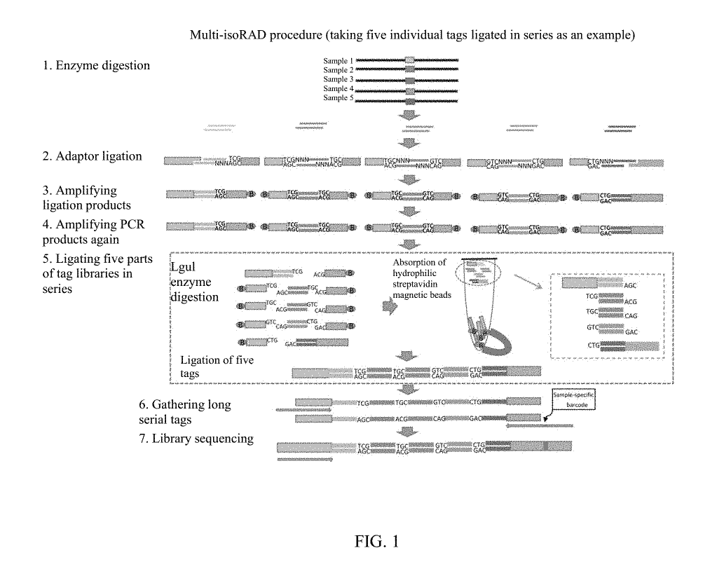

### DETAILED DESCRIPTION

The present embodiment establishes a construction method for serial sequencing libraries of RAD tags (abbreviated as serial tag sequencing technology or Multi-isoRAD technology), which can be applied to a paired-end sequencing platform.

A construction method for serial sequencing libraries of RAD tags in the present embodiment is completed in accordance with the following steps (taking five individual tags ligated in series as an example):

1) Preparing Five Parts of Genomic DNAs of Biological Samples for Performing Enzyme Digestion Reactions:

extracting biologic genomic DNAs and preserving it at 4° C. for standby; performing enzyme digestion reactions on five parts of samples' DNA by using endonuclease respectively to obtain five parts of enzyme-digested fragments, where DNA 5′ ends in, the generated tags have three-base overhangs.

The endonuclease can be selected from the IIB type restriction endonuclease and/or Mrr-like enzyme; the IIB type restriction endonuclease includes but is not limited to BsaXI, BcgI, BaeI, AguI, AlfI or CspCI; and the Mrr-like enzyme includes but is not limited to FspEI, MspJI, LpnPI, AspBHI, RIaI or SgrTI. Two types of enzymes have the featured of generating cleavage on the upstream and downstream of the recognition site and generate isolength tags (33-35 bp) with cohesive ends. An enzyme digestion system is 15 μL, which includes 200 ng of genomic DNA and 1 U of endonuclease (NEB), 1×cutsmart, and the reaction is preserved at 37° C. for 45 mins.

2) Designing Adaptors with Cohesive Ends and that are Ligated with the Tags:

Performing the ligation with the above five parts of the enzyme digestion reactions, and the adaptors contain restriction enzyme sites of SapI, featured base sequences for serial ligation of RAD tags, and universal sequences for the binding of amplification primers. The sequential ligations of N groups of enzyme digested fragments are determined according to the added adaptors.

In the present embodiment, the featured base sequence refers to a combination of three bases. A principle to follow is that three bases on the Adaptor Ada1b and three bases on the Adaptor Ada2a perform complementary pairing, three bases on the Adaptor Ada2b and three bases on the Adaptor Ada3a perform complementary pairing, three bases on the Adaptor Ada3b and three bases on the Adaptor Ada4a perform complementary pairing, and three bases on the Adaptor Ada4b and three bases on the Adaptor Ada5a perform complementary pairing, to ensure the sequential serial ligation of the enzyme-digested fragments. For example, three bases on the Adaptor Ada1b are 5′-CGA-3′, and three bases on the Adaptor Ada2a are 5′-TCG-3′ following a complementary pairing principle.

The restriction enzyme sites of SapI are

5′...GCTCTTC(N)₁^{▾}...3′
3′...CGAGAAG(N)₄_{_{▴}}...5′

In the present embodiment, a three-base featured sequence is designed on the 5′ end of the recognition site CGAGAAG; the featured sequence can form the 5′ protruding cohesive end after cleavage; and the tags are ligated in series by means of complementary pairing of the protruding cohesive ends on five pairs of adaptors.

Because the 5′ ends of the enzyme-digested fragments obtained in step 2) have three-base overhangs, five pairs of adaptors are designed in the present embodiment; the 3′ ends of the Adaptors have three combined bases, which enables five groups of different ligation reactions to be conducted to obtain five parts of ligation products. The adaptors used by five tags are shown in Table 1.

The combined bases are NNN. N is a combined base and represents any one of four bases: A, G, C and T. The generated tags after the digestion of BsaXI have three cohesive ends of random three bases. Therefore, three combined bases are designed on the adaptors in such a way that the adaptors can be ligated with the tags according to the complementary nature of the cohesive ends.

A ligation reaction system is 20 μL, including 10 μL of enzyme-digested fragments in step 1), and 200 U of T4 DNA ligation enzymes (NEB), 1×T4 Ligase Buffer, 4 uM AdaA, 4 uM AdaB, and 10 mM ATP, and they preserve the ligation reaction at 16° C. for 1 h.

TABLE 1
Adaptor combinations for the five tag positions

| Tag Positions | AdaA | AdaB |
|---|---|---|
| 1 | Ada1a | Ada1b |
| 2 | Ada2a | Ada2b |
| 3 | Ada3a | Ada3b |
| 4 | Ada4a | Ada4b |
| 5 | Ada5a | Ada5b |

As shown in Table 1, five pairs of adaptors are Ada1a and Ada1b, Ada2a and Ada2b, Ada3a and Ada3b, Ada4a and Ada4b, and Ada5a and Ada5b. Each Adaptor consists of two nucleotide fragments, where the two nucleotide fragments that form Ada1a have the sequences of SEQ ID NO: 1. and SEQ ID NO: 2; the two nucleotide fragments that form Ada1b have the sequences of SEQ ID NO: 3 and SEQ ID NO: 4; the two nucleotide fragments that form Ada2a have the sequences of SEQ ID NO: 5 and SEQ ID NO: 6; the two nucleotide fragments that form Ada2b have the sequences of SEQ ID NO: 7 and SEQ ID NO: 8; the two nucleotide fragments that form Ada3a have the sequences of SEQ ID NO: 9 and SEQ ID NO: 10; the two nucleotide fragments that form Ada3b have the sequences of SEQ ID NO: 11 and SEQ ID NO: 12; the two nucleotide fragments that form Ada4a have the sequences of SEQ ID NO: 13 and SEQ ID NO: 14; the two nucleotide fragments that form Ada4b have the sequences of SEQ ID NO: 15 and SEQ ID NO: 16; the two nucleotide fragments that form Ada5a, have the sequences of SEQ ID NO: 17 and SEQ ID NO: 18; and the two nucleotide fragments that form Ada5b have the sequences of SEQ ID NO: 19 and SEQ ID NO: 20. Five pairs of adaptors have design features: the restriction enzyme sites of SapI, the featured base sequences for the serial ligation of RAD tags, and the universal sequence for the binding of amplification primers in the adaptor sequences. However, a base mutation is designed on the enzyme digestion sites of SapI in Ada1a and Ada5b, which cannot be subjected to enzyme digestion. Therefore, when enzyme digestion is performed on the PCR products of five mixed tags by using the SapI enzyme (NEB), the universal sequence of adaptors and primers on the Ada1b and Ada5a, Ada2a and Ada2b, Ada3a and Ada3b, and Ada4a and Ada4b are excised, and the three-base featured sequences than cohesive ends on both sides of the five tag fragments. Sequential head-to-tail ligation of the five tags is performed according to complementary pairing of the featured sequences, i.e., Ada1b end is ligated with Ada2a end, Ada2b end is ligated with Ada3a end, Ada3b end is ligated with Ada4a end, and Ada4b end is ligated with Ada5a end, to form serial tags; Namely, Ada1b end is ligated with Ada2a end, Ada2b end is ligated with Ada3a end, Ada3b end is ligated with Ada4a end and Ada4b end is ligated with Ada5a end, to form serial tags; and the universal sequence of Adaptor ends of Ada1a and Ada5b on the serial tags is still reserved to provide a primer bonding point for the next amplification and gathering of serial tags.

The two nucleotide sequences that form Ada1a are

(SEQ ID NO: 1)
5′-ACACTCTTTCCCTACACGACGCTGTTCCGATCTNNN-3′
and
(SEQ ID NO: 2)
5′-AGATCGGAACAGC-3′.

The nucleotide sequences of Ada1b are

(SEQ ID NO: 3)
5′-GTGACTGGAGTTCAGACGTGTGCTCTTCACGANNN-3′
and
(SEQ ID NO: 4)
5′-TCGTGAAGAGCAC-3′.

The nucleotide sequences of Ada2a are

(SEQ ID NO: 5)
5′-ACACTCTTTCCCTACACGACGCTCTTCATCGNNN-3′
and
(SEQ ID NO: 6)
5′-CGATGAAGAGCGT-3′.

The nucleotide sequences of Ada2b are

(SEQ ID NO: 7)
5′-GTGACTGGAGTTCAGACGTGTGCTCTTCAGCANNN-3′
and
(SEO ID NO: 8)
5′-TGCTGAAGAGCAC-3′.

The nucleotide sequences of Ada3a are

(SEQ ID NO: 9)
5′-ACACTCTTTCCCTACACGACGCTCTTCATGCNNN-3′
and
(SEQ ID NO: 10)
5′-GCATGAAGAGCGT-3′.

The nucleotide sequences of Ada3b are

(SEQ ID NO: 11)
5′-GTGACTGGAGTTCAGACGTGTGCTCTTCAGACNNN-3′
and
(SEQ ID NO: 12)
5′-TCGTGAAGAGCAC-3′.

The nucleotide sequences of Ada4a are

(SEQ ID NO: 13)
5′-ACACTCTTTCCCTACACGACGCTCTTCAGTCNNN-3′
and
(SEQ ID NO: 14)
5′-GACTGAAGAGCGT-3′.

The nucleotide sequences of Ada4b are

(SEQ ID NO: 15)
5′-GTGACTGGAGTTCAGACGTGTGCTCTTCACAGNNN-3′
and
(SEQ ID NO: 16)
5′-CTGTGAAGAGCAC-3′.

The nucleotide sequences of Ada5a are

(SEQ ID NO: 17)
5′-ACACTCTTTCCCTACACGACGCTCTTCACTGNNN-3′
and
(SEQ ID NO: 18)
5′-CAGTGAAGAGCGT-3′.

The nucleotide sequences of Ada5b are

(SEQ ID NO: 19)
5′-GTGACTGGAGTTCAGACGTGTGCTGTTCCGATCTNNN-3′
and
(SEQ ID NO: 20)
5′-AGATCGGAACAGC-3′.

3) Amplifying Ligation Products and Gathering Tags:

performing PCR amplification on the five parts of ligation products obtained in step 2) by using a combination of different biotin primers and general primers; gathering the enzyme-digested fragments ligated with the adaptors; and amplifying to obtain the five parts of gathered PCR products.

The primer combinations have nucleotide sequences of SEQ ID NO: 21, SEQ ID NO: 22, SEQ ID NO: 23 and SEQ ID NO: 24. The primer combinations have design features with the following: selection of the primer combinations corresponding to the Adaptor combinations in step 2); as shown in Table 2, the enzyme-digested fragments ligated with the Adaptor 1 are amplified using primers Prim1 and BioPrim1; the, enzyme-digested fragments ligated With the Adaptors 2, 3 and 4 are amplified using primers BioPrim1 and BioPrim2; and the enzyme-digested fragments ligated with the Adaptor 5 are amplified using primers BioPrim1 and Prim2.

Namely, the universal sapience of the adaptor excised by the SapI enzyme is combined with the biotin during amplification; thus, the redundant fragments could be separated from the target tags using magnetic bead purification, which will achieve a higher efficiency of serial ligation of the tags

A PCR reaction system is 50 μL, including a reaction template of 18 μL, 8 uM PrimerA, 8 uM PrimerB, 12 mM dNTPs (NEB), and 0.8 U Phusion high-fidelity DNA polymerase (NEB), 1×HF buffer. The PCR reaction is conducted using the following conditions: 16 cycles of 98° C. for 5 s, 60° C. for 20 s and 72° C. for 10 s, as well as a final extension of 10 min at 72° C.

The amplified PCR products are checked through an 8% polyacrylamide gel, and the size of each amplified product is approximately 100 bp. The target band is excised from the gel, and the DNA is diffused from the gel in nuclease-free water for 6-12 h at 4° C. The collected products are amplified attain with the above method. Amplification is performed for 4-8 cycles. Five parts of amplified products are equally mixed and purified using the Qiagen MinElute PCR kit to remove redundant primers, Phusion enzyme, dNTP and other components, to avoid influencing the subsequent reactions.

TABLE 2
Primer combinations for the five tag positions

| Tag Positions | Primer A | Primer B |
|---|---|---|
| 1 | Prim1 | BioPrim2 |
| 2 | BioPrim1 | BioPrim2 |
| 3 | BioPrim1 | BioPrim2 |
| 4 | BioPrim1 | BioPrim2 |
| 5 | BioPrim1 | Prim2 |

A nucleotide sequence of Prim1 is

| | |
|---|---|
| 5′-ACACTCTTTCCCTACACGACGCT-3′. | (SEQ ID NO: 21) |

A nucleotide sequence of Prim2 is

| | |
|---|---|
| 5′-GTGACTGGAGTTCAGACGTGTGCT-3′. | (SEQ ID NO: 22) |

A nucleotide sequence of BioPrim1 is (biotin)

| | |
|---|---|
| 5′-ACACTCTTTCCCTACACGACGCT-3′. | (SEQ ID NO: 23) |

A nucleotide sequence of BioPrim2 is (biotin) 5′-GTGACTGGAGTTCAGACGTGTGCT-3′ (SEQ ID NO: 24).

4) Ligating the Five Parts of the Tag Libraries in Series:

performing enzyme digestion on the mixed and purified five parts of PCR products by using the SapI enzyme to excise universal adaptors and primers sequences on both ends of each enzyme-digested fragment, and the featured base sequences have formed cohesive ends, which enable the five parts of the PCR products to ligate in series; and the sequential ligation of the five parts of the tag libraries was based on the complementary pairing of the featured sequences on the five pairs of adaptors.

An enzyme digestion system is 30 μL, including 10 μL of the above mixed and purified PCR products (including 100-300 ng of the PCR product), 2 U of SapI enzyme (NEB) and 30 mM ATP, 1×Tango buffer. The enzyme digestion reaction is preserved at 37° C. for 30 min.

During this period, Streptavidin magnetic beads are prepared: gently shaking up streptavidin magnetic beads (NEB); absorbing 10 μL into a micro centrifuge tube and then applying a magnet to discard the supernatant; suspending the streptavidin magnetic beads using 20 μL 1×cutsmart buffer twice and discarding the supernatant to obtain balanced NEBs for later use.

Thirty microliters of enzyme digestion products are added to the above balanced NEB, and we incubate the mixture at room temperature for 5 mins with occasional agitation using a pipette. Apply a magnet and transfer the supernatant to a new tube. Add 200 U T4 DNA ligase to the supernatant, and incubate at 16° C. for 45 min to obtain the serial tag libraries.

The products are checked through 8% polyacrylamide gel, and the size of each ligation product is approximately 244 bp. The target band is excised from the gel, and the ligated product is diffused from the gel in nuclease-free water for 6-12 h at 4° C.

5) Performing PCR Amplification, Gathering Serial Tags and Introducing Library-Specific Barcode

To ensure that the serial libraries structure of the RAD tags is compatible with the sequencing platform, the primer Barcode is further used to amplify the serial tags; and barcodes are introduced for constructing the sequencing libraries, to have a sequencing primer binding site that is compatible with a next-generation sequencing platform.

A PCR amplification reaction system is 50 μL, including 7.5 μL of the ligation products in step 4), 5 uM Slx-Primer3, 5 uM Slx-Index Primer, 12 mM dNTPs (NEB), 0.8 U Phusion high-fidelity DNA polymerase (NEB), and 1×HF buffer. The PCR reaction is conducted using the following conditions: 16 cycles of 98° C. for 5 s, 60° C. for 20 s and 72° C. for 10 s, as well as a final extension of 10 min at 72° C.

The PCR amplification products are checked through 8% polyacrylamide gel, and the size of the target product is approximately 299 bp, The target band is excised from the gel, and the PCR products are diffused from the gel in nuclease-free water for 6-12 h at 4° C. Then, the gathered PCR products are purified with the Qiagen MinElute PCR product purification kit. Then, the library was subjected to Illumina HiSeq2500 sequencing (PE150).

A nucleotide sequence of Primer3 is

(SEQ ID NO: 25)
5′-AATGATACGGCGACCACCGAGATCTACACTCTTTCCCTACACGACGC
T-3′.

A nucleotide sequence of Index Primer is

5′-CAAGCAGAAGACGGCATACGAGATNNNNNNGTGACTGGAGTTCAGACGTGTGCTCTTCCGATCT-3′ (SEQ ID NO: 26), where NNNNNN can be changed according to different Barcode sequences.

6) Data Analysis:

(1) performing quality filtering on raw data obtained by Illumina sequencing to remove any sequences with ambiguous basecalls (N) and excessive low-quality positions (>5 bases with quality score <10)

(2) dividing the serial sequences according to the positions of the single tag and extracting the tags that contain the restriction sites from the five samples libraries

(3) performing data analysis on the tag sequences of five samples using bioinformatic software (such as open acquisition software Stacks, RAD typing) to analyze the SNP sites or methylation information.

The library construction method established in the present embodiment provides a method for serial sequencing of isolength RAD tags on a next-generation platform, and it allows the controllability of the number of serial tags during ligation. At the same time, the configuration of the concatenated tags is highly flexible, and it can be defined by users to work with a desired combination of samples and/or restriction enzymes to suit specific research purposes (SNP genotyping or quantification of the DNA methylation level). The technology inherits the advantages of isolength RAD technology and the current mainstream paired-end sequencing method, and it provides an efficient and flexible means for the screening and detection of genome-wide genetic variations and epigenetic variation.

### EMBODIMENT 1

The library construction method in the present embodiment is described in detail below by taking Patinopccten Yessoensis as experimental material for the serial sequencing of different types of tag libraries as an example. For the reagents, reaction conditions, and other factors used in the present embodiment, those skilled in the art can make a choice in the prior art according to a technical solution in the present embodiment, and it is not limited to a specific embodiment in the present embodiment.

1. Extracting Scallop Genomic DNA

taking approximately 0.1 g of adductor muscle of one sample of Patinopecten Yessoensis; adding it to 500 μL of STE buffer, which includes NaCI: 100 mmol/L; EDTA:1 mmol/L, pH=8.0: Tris-HCl, 10 nmol/L, pH=8.0; shearing; adding 50 μL of 10% SDS (sodium dodecyl sulfate) and 5 μL of proteinase K (20 mg/mL); digesting in a water bath of 56° C. until the tissue fragments are completely pyrolyzed to obtain clear lysate; adding isovolumic saturated phenol (250 μL) and chloroform/isoamyl alcohol (volume ratio of 24:1) (250 μL); extracting three times; absorbing the supernatant: adding isovolumic chloroform/isoamyl alcohol (24:1) (500 μL); extracting once; absorbing the supernatant; adding 1/10 volumic CH3COONa (3 mol/L, pH 5.2)(50 μL) and 2 times the volume of 100% ethyl alcohol (1000 ul) stored at −20° C.; shaking up slowly; precipitating for 30 min at −20° C.; then centrifuging for 10 min at 12000 rpm; precipitating the nucleic acid at the tube bottom; washing the precipitate with 70% ethanol (1000 ul) and drying until the ethanol is completely volatilized; adding 100 ul of sterile water and 1-2 μL RNase A (ribonuclease); and storing in a refrigerator at 4° C. for standby.

2. Digesting Scallop Genomic DNA

selecting three IIB type restriction endonucleases (BsaXI, BcgI and BaeI) and two Mrr-like enzymes (FspEI and MspJI) for enzyme digestion of genomic DNA to obtain different types of five enzyme digestion products,

where an enzyme digestion system is 15 μL, which includes 200 mg of genomic DNA and 1 U of endonuclease (NEB), 1×cutsmart; and the reaction is preserved at 37° C. for 45 mins.

3. Ligating the Enzyme-Digested Fragment with Adaptors as Bonding Points of Amplification Primers

Ligating the five parts of the enzyme digestion products with different adaptor combinations as shown in Table 3, to obtain five parts of ligation products.

A ligation reaction system is 20 μL, including the enzyme digestion products of 10 μL in step 2) and the T4 DNA ligation enzymes (NEB) of 200 U, 1×T4 Ligase Buffer, 4 uM Slx-AdaA, 4 uM Slx-AdaB, and 10 mM ATP. The reaction is preserved at 16° C. for 1 h.

TABLE 3
Adaptor Combinations for the Five Parts of Enzyme
Digestion Products in Embodiment

| Tag Positions | Six-AdaA | Six-AdaB |
|---|---|---|
| Tag 1 (BsaXI) | Ada1a | Ada1b |
| Tag 2 (Bcg I) | Ada2a | Ada2b |
| Tag 3 (Bae I) | Ada3a | Ada3b |
| Tag 4 (FspEI) | Ada4a | Ada4b |
| Tag 5 (MspJI) | Ada5a | Ada5b |

4. Performing PCR Amplification on the Enzyme-Digested Fragment Ligated with the Adaptors and Gathering Tags

performing PCR amplification on the five parts of ligation products obtained in step 3 using the combination of primers provided in Table 4; and gathering the enzyme-digested fragments to obtain live parts of PCR products.

A PCR amplification reaction system is 50 μL, including a reaction template of 18 μL, 8 uM PrimerA, 8 uM PrimerB, 12 mM dNTPs, and 0.8 U Phusion high-fidelity DNA polymerase (NEB), with 1×HF buffer. The PCR reaction is conducted using the following conditions: 16 cycles of 98° C. for 5 s, 60° C. for 20 s and 72° C. for 10 s, and then, there is a final extension of 10 min at 72° C. p PrimerA is (5′-ACACTCTTTCCCTACACGACGCT-3′), and PrimerB is (5′-GTGACTGGAGITCAGACGIGTGCT-3′).

TABLE 4
Primer Combinations for the five tag positions in Embodiment 1

| Tag Positions | Primer A | Primer B |
|---|---|---|
| Tag 1 (BsaXI) | Prim1 | BioPrim2 |
| Tag 2 (Bcg I) | BioPrim1 | BioPrim2 |
| Tag 3 (Bae I) | BioPrim1 | BioPrim2 |
| Tag 4 (FspEI) | BioPrim1 | BioPrim2 |
| Tag 5 (MspJI) | BioPrim1 | Prim2 |

Five parts of PCR products are checked through an 8% polyacrylamide gel, and the size of each amplified product is approximately 100 bp. The target band is excised from the gel, and the DNA is diffused from the gel in nuclease-free water for 6-12 h at 4° C. The collected five parts of the PCR products are amplified again following the above method. Amplification is performed for 7 cycles. The five parts of amplified products are mixed in equal volume and purified using the Qiagen MinElute PCR kit to obtain one part of PCR purified product.

5. Enzyme Digestion and Ligation

performing enzyme digestion on the mixed PCR products using the SapI enzyme and enabling the tag libraries to be ligated in series. An enzyme digestion system is 30 μL, which includes the PCR purified products of 10 μL in step 4, 2 U SapI enzyme (NEB), 30 ATP and 1×Tango buffer. The enzyme digestion reaction is preserved at 37° C. for 30 mins. Then, the 30 μL of digested products are added to the prepared Streptavidin magnetic beads (NEB), and the reaction is preserved at room temperature for 5 mins with occasional agitation using a pipette. After 5 mins, the enzyme digestion products are placed on a magnet and stand for 2 min and, then, are transferred the supernatant to a new micro centrifuge tube. Add 200 U T4 DNA ligase to the supernatant and incubate at 16° C. for 45 min to obtain sequentially the tags in series.

Step of preparing Streptavidin magnetic beads: gently shaking up streptavidin magnetic beads (NEB); absorbing 10 μL into a micro centrifuge tube and then applying a magnet to discard the supernatant; then, carefully washing the streptavidin magnetic beads using 20 μL 1×cutsmart buffer twice and discarding the supernatant to obtain balanced NEBs for later use.

After 30 mins, the serial tag products are checked through an 8% polyacrylamide gel, and the size of the ligation product is approximately 244 bp. The target band is excised from the gel, and the ligated product is diffused from the gel in nuclease-free water for 6-12 h at 4° C.

6. Performing PCR Amplification and Introducing the Library-Specific Barcode

The serial tag products are timber amplified using the barcode primers, and the universal sequences required fin the Illumina platform sequencing are introduced.

A PCR reaction system is 50 μL, which includes 7.5 μL of ligation products, 5 uM Slx-Primer3, 5 uM Slx-Index. Primer, 12 mM dNTPs, 0.8 U Phusion high-fidelity DNA polymerase (NEB), and 1×HF buffer. The PCR reaction is conducted using the following conditions: 16 cycles of 98° C. for 5 s, 60° C. for 20 s and 72° C. for 10 s, and then, there is a final extension of 10 min at 72° C. Two tubes of products are amplified in parallel.

A sequence of Slx-Primer3 is

(5′-AATGATACGGCGACCACCGAGATCTACACTCTTTCCCTACACGACG
CT-3′);

A sequence of Slx-Index Primer is

(5′-CAAGCAGAAGACGGCATACGAGATNNNNNNGTGACTGGAGTTCAGACGTGTGCTCTTCCGATCT-3′, where NNNNNN can be changed according to different Barcode sequences.

The PCR amplification products are checked through an 8% polyacrylamide gel, and the size of the target product is about 299 bp. The PCR products are purified using the Qiagen MinElute PCR product purification kit. Then, the library was subjected to Illumina HiSeq2500 sequencing (PE150).

7. Data Analysis

1) performing quality filtering on raw data obtained by Illumina sequencing to remove any sequences with ambiguous basecalls (N) and excessive low-quality positions (>5 bases with a quality score of <10). 98.9% of the sequencing reads were retained as high-quality reads for further analyses.

2) dividing the serial sequences according to the positions of the single tag and extracting the tags that contain the restriction sites from the five sample libraries. 90.3%, 91.4% 90.1%, 90.0% and 92.2% of the HQ reads contained the target restriction sites in the BsaXI, BcgI, BaeI, FspEI and MspJI library, respectively. The extraction rate of the tags that contain the restriction sites were more than 90% in different types of libraries, which indicates that the tag libraries can be sequentially ligated in series, as expected,

3) performing data analysis on the tag sequences of five samples using bioinformatic software: The 2b-RAD library data were processed using the RAD-typing software to obtain the number of restriction sites and the SNPs information. 93.15% of the total unique sites that are predicted from reference genomes can be detected, 96.02% of which was in agreement with the standard single-tag sequencing data generated using the standard 2b-RAD protocol. Genotype calls of common loci achieved 99.2% genotype concordance compared with the single-tag sequencing data. MethylRAD library data were processed using CD-HIT software to obtain methylated sites and an abundance of representative tags, i.e., the methylation level of the site. 130162 FspEI methylated sites are obtained, including 90.67% of the sites in the single tag library, and 260545 MspJI methylated tags, including 91.4% of the sites in the single tag library. The correlation of sequencing depth across the methylated sites between the serial sequencing library and single-tag sequencing library achieved more than 0.90.

In summary, the result shows that the multienzyme serial sequencing library construction method allows researchers to perform a high-resolution genome scan to detect both genetic and epigenetic variations in the same sample. The current protocol described here addresses the issue with the original isoRAD protocol in that it cannot be adapted for cost-effective PE sequencing. It also provides researchers more power and flexibility in devising effective library configurations to meet specific research purposes.

TABLE 5
Primer Sequences Involved in the Present
Embodiment

| Adap- | |
|---|---|
| tor and | |
| Primer | |
| Names | Adaptor and Primer Sequences |
| Slx- | 5′-ACACTCTTTCCCTACACGACGCTGTTCCGATCTNNN- |
| Ada1a | 3′ |
| | (3′ AminoC6)3′-CGACAAGGCTAGA-5′ |
| Slx- | 5′-GTGACTGGAGTTCAGACGTGTGCTCTTCACGANNN-3′ |
| Ada1b | (3′AminoC6)3′-CACGAGAAGTGCT-5′ |
| Slx- | 5′-ACACTCTTTCCCTACACGACGCTCTTCATCGNNN-3′ |
| Ada2a | (3′ AminoC6)3′-TGCGAGAAGTAGC-5′ |
| Slx- | 5′-GTGACTGGAGTTCAGACGTGTGCTCTTCAGCANNN-3′ |
| Ada2b | (3′ AminoC6)3′-CACGAGAAGTCGT-5′ |
| Slx- | 5′-ACACTCTTTCCCTACACGACGCTCTTCATGCNNN-3′ |
| Ada3a | (3′ AminoC6)3′-TGCGAGAAGTACG-5′ |
| Slx- | 5′-GTGACTGGAGTTCAGACGTGTGCTCTTCAGACNNN-3′ |
| Ada3b | (3′ AminoC6)3′-CACGAGAAGTCYG-5′ |
| Slx- | 5′-ACACTCTTTCCCTACACGACGCTCTTCAGTCNNN-3′ |
| Ada4a | (3′ AminoC6)3′-TGCGAGAAGTCAG-5′ |
| Ada4b | 5′-GTGACTGGAGTTCAGACGTGTGCTCTTCACAGNNN-3′ |
| | (3′ AminoC6)3′-CACGAGAAGTGTC-5′ |
| Ada5a | 5′-ACACTCTTTCCCTACACGACGCTCTTCACTGNNN-3′ |
| | (3′ AminoC6)3′-TGCGAGAAGTGAC-5′ |
| Ada5b | 5′-GTGACTGGAGTTCAGACGTGTGCTGTTCCGATCTNNN- |
| | 3′ |
| | (3′ AminoC6)3′-CGACAAGGCTAGA-5′ |
| Prim 1 | 5′-ACACTCTTTCCCTACACGACGCT-3′ |
| Prim 2 | 5′-GTGACTGGAGTTCAGACGTGTGCT-3′ |
| BioPrim | (biotin) 5′-ACACTCTTTCCCTACACGACGCT-3′ |
| 1 | |
| BioPrim | (biotin) 5′-GTGACTGGAGTTCAGACGTGTGCT-3′ |
| 2 | |
| primer | 5′-AATGATACGCCGACCACCGAGATCTACACTCTTTCC |
| 3 | CTACACGACGCT-3′ |
| Slx- | 5′-CAAGCAGAAGACGGCATACGAGATNNNNNNGTGAC |
| Index | TGGAGTTCAGACGTGTGCTCTTCCGATCT-3 |
| Primer | |

What is claimed is:

1. A construction method for serial sequencing libraries of RAD tags, including the following steps:
1) enzyme digestion: conducting an enzyme digestion reaction with N samples of genomic DNA using selected endonucleases to obtain N its of enzyme-digested fragments, where N is an integer greater than 2;
2) adaptor ligation: ligating N parts of enzyme-digested fragments with, adaptors, Le., N pairs of adaptor pairs are designed to obtain N parts of ligated products, and the adaptors contain restriction enzyme sites of SapI, featured base sequences for the serial ligation of RAD tags, and universal sequences for the binding of amplification primers, and the sequential ligation of N groups of enzyme-digested fragments are determined according to the added adaptors;
3) amplification of ligated products: conducting PCR amplification on the N parts of the ligated products obtained in step 2) using a different combination of biotin primers and general primers; collecting PCR products by gel; amplifying 4-8 cycles using the same method to obtain N parts of enriched PCR products; and equally mixing the N parts of enriched PCR products and purifying;
4) serial ligation of tag libraries: conducting enzyme digestion on the mixed and purified N parts of PCR products using the SapI enzyme to excise universal adaptor and primer sequences on both ends of each enzyme-digested fragment, and the featured base sequences form cohesive ends that enable the N parts of the PCR products to ligate in series; and the sequential ligation of the N parts of the tag libraries is based on the complementary pairing of the featured sequences on the adaptors.
5) amplification of ligated serial tags: purifying the long serial tags through a gel and then conducting PCR amplification using the barcode primers to construct the libraries of serial RAD tags
6) library sequencing: sequencing the libraries of serial tags on the Illumina sequencing platform.

2. The construction method for the serial sequencing libraries of RAD tags according to claim 1, where the endonuclease in step 1) is one or more of IIB type restriction endonuclease and Mrr-like family of methylation-dependent restriction enzymes.

3. The construction method for the serial sequencing libraries of RAD tags according to claim 1, where the adaptors in step 2) have the following design features: five pairs of adaptors are designed; the five pairs of adaptors are Ada1a and Ada1b, Ada2a and Ada2b, Ada3a and Ada3b, Ada4a and Ada4b, and Ada5a and Ada5b, each adaptor consists of two nucleotide fragments: a base mutation is designed on the restriction enzyme sites of SapI in Adaptors Ada1a and Ada5b, which cannot be subjected to enzyme digestion; when enzyme digestion is conducted on the PCR products of five mixed tags by using the SapI enzyme, universal sequence of adaptors and primers on the Ada1b and Ada5a, Ada2a and Ada2b, Ada3a and Ada3b, and Ada4a and Ada4b are excised, and the three-base featured sequences form cohesive ends on both sides of the five tag fragments; sequential head-to-tail ligation of the five tags is performed according to complementary pairing of the featured sequences, i.e., Ada1b end is ligated with Ada2a end, Ada2b end is ligated with Ada3a end, Ada3b end is ligated with Ada4a end, and Ada4b end is ligated with Ada5a end, to form serial tags; and the universal sequence of Adaptors Ada1a and Ada5b on the serial tags is still reserved, thereby providing a primer bonding point for the next amplification and gathering of serial tags.

4. The construction method for the serial sequencing libraries of RAD tags according to claim 3, where in step 2), two nucleotide fragments that form Ada1a have the sequences of SEQ ID NO: 1 and. SEQ ID NO: 2; the two nucleotide fragments that form Ada1b have the sequences of SEQ ID NO: 3 and SEQ ID NO: 4; the two nucleotide fragments that form Ada2a have the sequences of SEQ ID NO: 5 and SEQ ID NO: the two nucleotide fragments that form Ada2b have the sequences of SEQ ID NO: 7 and SEQ ID NO: 8; the two nucleotide fragments that form Ada3a have the sequences of SEQ ID NO: 9 and SEQ ID NO: 10; the two nucleotide fragments that form Ada3b have the sequences of SEQ ID NO: 11 and SEQ ID NO: 12; the two nucleotide fragments that form Ada4a have the sequences of SEQ ID NO: 13 and SEQ ID NO; 14; the two nucleotide fragments that form Ada4b have the sequences of SEQ ID NO: 15 and SEQ ID NO: 16; the two nucleotide fragments that form Ada5a have the sequences of SEQ ID NO: 17 and SEQ ID NO: 18; and the two nucleotide fragments that form Ada5b have the sequences of SEQ ID NO: 19 and SEQ ID NO: 20.

5. The construction method for the serial sequencing libraries of RAD tags according to claim 4, where in step 3), the option of a combination of biotin primers and general primers that correspond to the adaptor pairs in step 2); the enzyme-digested fragments ligated with the adaptors 1 are amplified using primers Prim1 and BioPrim1; the enzyme-digested fragments ligated with adaptors 2, 3 and 4 are amplified using primers BioPrim1 and BioPrim2; and the enzyme-digested fragments ligated with adaptor 5 are amplified using primers BioPrim1 and Prim2.

6. The construction method for the serial sequencing libraries of RAD tags according to claim 5, where the nucleotide sequence of the Prim1 is SEQ ID NO; 21; the nucleotide sequence of the Prim2 is SEQ ID NO: 22; the nucleotide sequence of the BioPrim1 is SEQ ID NO: 23; and the nucleotide sequence of the BioPrim2 is SEQ ID NO: 24.

7. The construction method for the serial sequencing libraries of RAD tags according to claim 6, where the nucleotide sequences of the primers in step 5) are SEQ ID NO: 25 and SEQ ID NO: 26.
